(19) United States
(12) Patent Application Publication
(10) Pub. No.: US 2010/0124739 A1
(43) Pub. Date: May 20, 2010

(54) Methods and Compositions for Diagnosing Pelvic Floor Dysfunction

(76) Inventor: John Klutke, Pacific Palisades, CA (US)

Correspondence Address: BRYAN CAVE LLP, 211 NORTH BROADWAY, SUITE 3600, ST. LOUIS, MO, 63102-2750, US

(21) Appl. No.: 12/270,926

(22) Filed: Nov. 14, 2008

(51) Int. Cl. C12Q 1/68

(52) U.S. Cl. 435/6

(57) ABSTRACT

The present invention relates to, among other things, methods and compositions for diagnosing and preventing pelvic floor dysfunction, genital prolapse, and similar medical conditions. For example, according to certain embodiments of the present invention, methods of diagnosing genital prolapse and/or pelvic floor dysfunction are provided. Such methods generally comprise (1) collecting a sample of a patient's uterosacral ligament tissue, (2) extracting total genomic DNA from the tissue, (3) amplifying a promoter region operably connected to a nucleic acid sequence encoding lysyl oxidase (LOX), lysyl oxidase like-1 (LOXL1), lysyl oxidase like-2 (LOXL2), lysyl oxidase like-3 (LOXL3), and/or lysyl oxidase like-4 (LOXL4), and (4) determining whether the promoter region comprises methylated CpG islands.

### FIELD OF THE INVENTION

The field of the present invention relates to methods and compositions for diagnosing and preventing a medical condition related to connective tissue failure or dysfunction, such as pelvic floor dysfunction, genital prolapse, and similar medical conditions.

### BACKGROUND OF THE INVENTION

Pelvic floor dysfunction is a “hidden epidemic” with profound functional consequences for individuals. A study in the United States indicated that approximately 11% of adult American women will have corrective surgery for the condition. Re-operation in these women is a common occurrence, which reinforces the need for improvements in the methods by which this condition may be avoided and/or treated.

It has been shown that elastin is a significant component of vaginal connective tissue. Connective tissue integrity depends on lysyl oxidase, an extracellular enzyme found in the extracellular matrix with collagen and elastin fibers. Lysyl oxidase catalyzes the initial step in crosslink formation in collagen and elastin fibers. At least four isoenzymes of lysyl oxidase (LOX) have been identified, namely, lysyl oxidase like-1 (LOXL1), lysyl oxidase like-2 (LOXL2), lysyl oxidase like-3 (LOXL3), and/or lysyl oxidase like-4 (LOXL4).

LOXL1 is notable because it appears to be specific to elastin metabolism. The crosslinking amino acids, desmosine and isodesmosine, formed through the action of LOXL1, are unique to elastin and have been used as a quantitative measure of elastin content. It is believed that elastin deficiency, perhaps caused by abnormal LOX expression, may play a role in the development of pelvic floor dysfunction, genital prolapse, and similar medical conditions.

In view of the foregoing, a need exists for methods and compositions for diagnosing and/or preventing medical conditions related to connective tissue failure or dysfunction, such as pelvic floor dysfunction, genital prolapse, and similar medical conditions.

### SUMMARY OF THE INVENTION

According to certain aspects of the present invention, methods of diagnosing a medical condition related to connective tissue failure or dysfunction are provided. Such methods generally comprise (1) extracting total genomic DNA from a specimen collected from a patient, (2) amplifying a promoter region operably connected to a nucleic acid sequence encoding a protein selected from the group consisting of lysyl oxidase (LOX), lysyl oxidase like-1 (LOXL1), lysyl oxidase like-2 (LOXL2), lysyl oxidase like-3 (LOXL3), and lysyl oxidase like-4 (LOXL4), and (4) determining whether the promoter region comprises methylated CpG islands.

According to additional aspects of the present invention, methods of preventing and ameliorating the effects of genital prolapse and/or pelvic floor dysfunction are provided. Such methods generally comprise determining whether a promoter region operably linked to a LOX-, LOXL1-, LOXL2-, LOXL3-, and/or LOX4-encoding sequence comprises methylated CpG islands and, if so, prescribing a preventative action to the patient. According to certain embodiments of the invention, a non-limiting example of a preventative action may be undergoing a cesarean section to deliver a child and foregoing a vaginal delivery.

According to further aspects of the present invention, kits for diagnosing a medical condition related to connective tissue failure or dysfunction, such as genital prolapse and/or pelvic floor dysfunction, are provided. According to certain embodiments, such kits may comprise PCR primers for amplifying a portion of a patient's genomic DNA that comprises a promoter region operably linked to a LOX-, LOXL1-, LOXL2-, LOXL3- and/or LOXL4-encoding sequence. In addition, such kits may comprise reagents and other tools for determining whether such promoter region comprises methylated CpG islands, such as, for example, DNA sequencing reagents and control DNA (or nucleic acid sequence data therefor).

### BRIEF DESCRIPTION OF THE FIGURES

The file of this patent application contains at least one drawing executed in color. Copies of any patent to issue from this application with color drawing(s) will be provided by the United States Patent and Trademark Office upon request and payment of the necessary fee.

FIG. 1 is a graphical representation of a methylated and non-methylated cytosine residue.

FIG. 2 is a graphical representation showing the effect of cytosine methylation on transcription factor binding and gene expression.

FIG. 3 is a flow chart outlining a non-limiting example of a process that may be followed to determine whether a promoter region operably linked to a LOX-, LOXL1-, LOXL2-, LOXL3-, and/or LOXL4-encoding sequence comprises methylated CpG islands.

FIG. 4 is a non-limiting example of a limited portion of nucleic acid sequence data related to a promoter region operably linked to a LOX-encoding sequence.

FIG. 5 is a graphical representation of the results of an assay to determine whether a promoter region operably linked to a LOX-encoding sequence in a plurality of test samples (and control samples) comprises methylated CpG islands.

### DETAILED DESCRIPTION OF THE INVENTION

The following will describe, in detail, several preferred embodiments of the invention. These embodiments are provided by way of explanation only, and thus, should not unduly restrict the scope of the invention. In fact, those of ordinary skill in the art will appreciate upon reading the present specification and viewing the present drawings that many variations and modifications of the invention may be employed, used and made without departing from the scope and spirit of the invention.

According to certain embodiments of the present invention, methods of diagnosing medical conditions related to connective tissue failure or dysfunction are provided. The medical condition will preferably be an elastin-related medical condition, such as genital prolapse, pelvic floor dysfunction, cervical incompetence, aortic aneurysm, or a predisposition to developing any of the foregoing. The methods of such embodiments generally comprise extracting total genomic DNA from a specimen collected from a patient. The invention provides that the total genomic DNA may be extracted from a variety of sources, such as uterosacral ligament tissue, cells collected through vaginal scraping, a patient's whole blood, or a combination of the foregoing.

The methods of the present invention further comprise amplifying a promoter region operably connected to a nucleic acid sequence encoding a lysyl oxidase (LOX)-related protein. The LOX-related protein may be lysyl oxidase (LOX), lysyl oxidase like-1 (LOXL1), lysyl oxidase like-2 (LOXL2), lysyl oxidase like-3 (LOXL3), and/or lysyl oxidase like-4 (LOXL4). As used herein, the term “operably connected” refers to the functional linkage between a promoter sequence and a LOX-encoding gene, such as a sequence encoding any of LOX, LOXL1, LOXL2, LOXL3, or LOXL4. The LOX, LOXL1, LOXL2, LOXL3, and LOXL4 enzymes are sometimes collectively referred to herein as the “LOX enzymes,” whereas the nucleic acid sequences encoding any of the LOX enzymes are collectively referred to herein as the “LOX-encoding sequences.” According to certain preferred embodiments, the invention provides that the promoter region that is operably connected to a LOX-encoding sequence comprises a nucleic acid sequence that is substantially similar to SEQ ID NO: 1 or, in certain embodiments, identical to SEQ ID NO: 1. Furthermore, the invention provides that such promoter region is, preferably, operably connected to a sequence encoding LOX or LOXL1, insofar as the invention provides that the reduction in the expression of these two particular enzymes is tightly correlated with elastin-related medical conditions, such as pelvic floor dysfunction, genital prolapse, and similar medical conditions. The promoter region operably connected to a LOX-encoding sequence may be amplified using any suitable PCR primers. Non-limiting examples of such PCR primers include the two primer sequences represented by SEQ ID NO: 2 and SEQ ID NO: 3.

It will be understood by those skilled in the art that two nucleic acid sequences are “substantially similar” when approximately 70% or more (preferably at least about 80%, and most preferably at least about 90% or 95%) of the nucleotides match over the defined length of the nucleic acid sequence. Sequences that are substantially homologous can be identified by comparing the sequences using readily accessible computer software, or in a Southern hybridization experiment under, for example, stringent conditions as defined for that particular system. Defining appropriate hybridization conditions for a particular system is within the skill of the art. It will be further understood by those skilled in the art that the phrase “at least substantially similar” refers to sequences that are “substantially similar” as described above or, alternatively, identical to one another.

The methods of the present invention further comprise determining whether the promoter region that is operably connected to a LOX-encoding sequence comprises methylated CpG islands. The term “CpG islands” refers to areas of a promoter region that are operably connected to a LOX-encoding sequence, which contains a relatively high frequency of CG dinucleotides (cytosine and guanine dinucleotides). More specifically, in certain embodiments, the invention further comprises determining whether the amplified (and preferably isolated portion of the) promoter region that is operably connected to a LOX-encoding sequence comprises methylated CpG islands. Any of various methods may be employed to determine whether such promoter region comprises methylated CpG islands. For example, such determination may be carried out by sequencing the promoter region that is operably connected to a LOX-encoding sequence derived from a patient (i.e. a test sample or a set of test sequence data).

Next, the test sequence data may be compared to a set of control sequence data. The control sequence data will preferably represent a nucleic acid sequence of a promoter region that is operably connected to a similar type of LOX-encoding sequence (also referred to herein as a “LOX promoter”) that does not contain any, or does not contain a biologically relevant amount of, methylated CpG islands. This comparison will reveal whether the test (patient's) LOX promoter contains methylated CpG islands, or significantly more methylated CpG islands than a representative LOX promoter of a control group. The control group may be a population of patients who, for example, have minimal or no apparent cystocele, rectocele or uterine prolapse on preoperative pelvic examination by a urogynecologist.

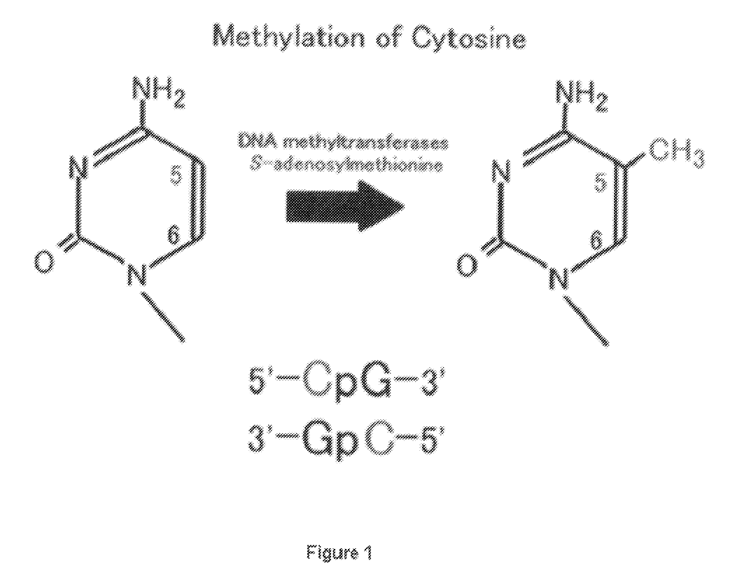

Any of various methods, whether currently-available or discovered hereafter, may be employed to determine whether a LOX promoter contains methylated CpG islands. More specifically, there are several methods of detecting 5-methylcytosines (which are illustrated in FIG. 1) or otherwise analyzing DNA methylation patterns, with the most common having sodium bisulfate treatment as a first step. Treatment of DNA with bisulfite ions (frequently in the form of sodium bisulfite) converts unmethylated cytosine to uracil, while leaving the 5-methylcytosines residues unmodified. These treated DNA sequences are recovered, desulfonated and typically amplified via polymerase chain reaction (PCR) techniques to increase the quantity of the DNA template for ease of analysis. Subsequent analysis of bisulfite-treated DNA sequences may be used to detect the quantity and/or position of uracil residues, which replace the unmethylated cytosines, and of the remaining cytosine residues, all of which would be methylated. A method for determining the methylation state of nucleic acids using bisulfite treatment is described in, for example, U.S. Pat. No. 6,017,704, which is hereby incorporated by reference in its entirety.

This subsequent analysis may be performed in a number of ways, namely, any sufficiently reliable and economical method capable of differentiating between single nucleotide polymorphisms. The amount or distribution of uracil residues produced by bisulfite treatment may be detected in various ways, including but not limited to sequencing or selective oligonucleotide hybridization. Additionally, an amplification reaction such as a polymerase chain reaction (PCR) may be performed on bisulfite-treated DNA sequences to aid in detection of the amount or distribution of uracil residues. Methylation-specific PCR (MSP) employs the bisulfite-treated DNA as the PCR template and uses methylation-specific primer pairs. Methylation-specific PCR is described in U.S. Pat. Nos. 5,786,146, 6,200,756, 6,017,704 and 6,265,171, each of which is incorporated herein by reference in its entirety. The resulting amplified DNA fragments may then be quantified by any method known to those with skill in the art, such as separation by molecular weight or charge, probe hybridization, or microarrays. Several representative methods of such analysis are described in U.S. Pat. Nos. 7,229,759 and 7,186,512, each of which is incorporated herein by reference in its entirety. Alternatively, the DNA region of interest, once treated with bisulfite and amplified with MSP, may be transformed into a plasmid vector. The nucleotide sequence of the plasmid DNA may then be determined using any number of sequencing methods, including but not limited to gel electrophoresis, pyrosequencing, or reversible terminator methods.

One alternative to bisulfite sequencing is methylated DNA immunoprecipitation (MeDIP). In MeDIP, isolated genomic DNA is fragmented (via digestion, sonification, or any other appropriate method) and denatured, then immunoprecipitated with antibodies that are specifically directed towards 5-methylcytosine. The resulting immune complexes are then isolated and their corresponding DNA regions may be amplified with PCR to allow for further analysis of the methylation patterns across a region of DNA.

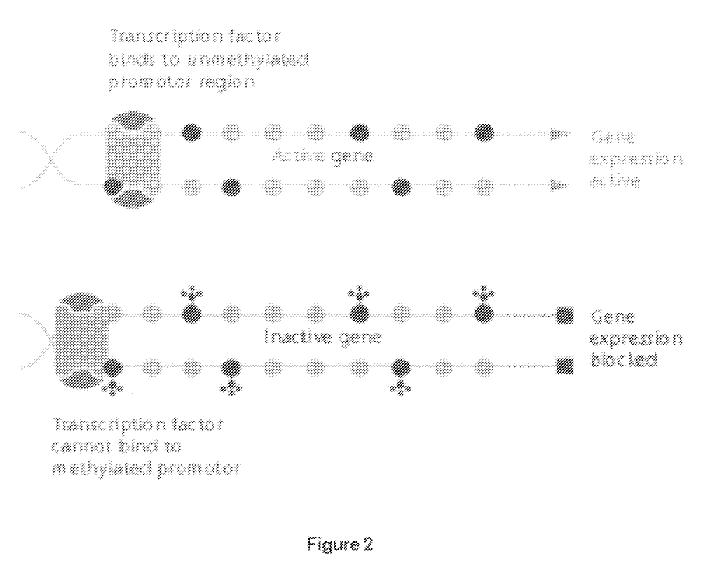

The invention provides that the presence of methylated CpG islands in a LOX promoter is indicative that the patient from which the LOX promoter was derived may have, or does have, a medical condition related to connective tissue failure or dysfunction, such as pelvic floor dysfunction, genital prolapse, and/or similar medical conditions. More particularly, and referring to FIG. 2, the invention provides that when the LOX promoter is not methylated, transcription factors may bind to or otherwise come into sufficient proximity of the LOX promoter, which is required for LOX expression. In contrast, when the LOX promoter is methylated (i.e., comprises methylated CpG islands), transcription factors may not bind to or are otherwise prevented from coming into sufficient proximity of the LOX promoter, which prevents or reduces LOX expression. The invention provides that the prevention or reduction in LOX expression, which is caused by the presence of methylated CpG islands in the LOX promoter, results in connective tissue failures or dysfunction. Such connective tissue failures or dysfunction will in turn cause pelvic floor dysfunctions, genital prolapse, and/or similar medical conditions.

According to additional aspects of the present invention, methods of preventing and ameliorating the effects of specific medical conditions are provided, such as genital prolapse and/or pelvic floor dysfunction. Such methods generally comprise determining whether a promoter region operably linked to a LOX-, LOXL1-, LOXL2-, LOXL3-, and/or LOX4-encoding sequence comprises methylated CpG islands and, if so, prescribing a preventative action to the patient. According to certain embodiments of the invention, a non-limiting example of a preventative action may be undergoing a cesarean section to deliver a child and foregoing a vaginal delivery.

According to further aspects of the present invention, kits for diagnosing a medical condition related to connective tissue failure or dysfunction, such as genital prolapse and/or pelvic floor dysfunction, are provided. According to certain embodiments, such kits may comprise PCR primers for amplifying a portion of a patient's genomic DNA that comprises a promoter region operably linked to a LOX-, LOXL1-, LOXL2-, LOXL3-, and/or LOXL4-encoding sequence. In addition, such kits may comprise reagents and other tools for determining whether such promoter region comprises methylated CpG islands, such as, for example, DNA sequencing reagents and control DNA (or nucleic acid sequence data therefor).

The following examples are provided to further illustrate the methods and compositions of the present invention. These examples are illustrative only and are not intended to limit the scope of the invention in any way.

### EXAMPLES

Methods.

Chemicals and Supplies. All chemicals used in the following examples were of molecular biology grade or higher and were purchased from Sigma-Aldrich Chemical Company (St. Louis, Mo.), unless otherwise stated. Molecular biology reagents were purchased from Promega (Madison, Wis.), Roche Molecular Biochemicals (Indianapolis, Ind.), and Life Technologies (Gaithersburg, Md.).

Tissue Collection and Processing. After Institutional Review Board approval, a total of 60 (31 subjects with prolapse and 29 controls) freshly isolated uterosacral ligament samples were collected. Subjects with prolapse had grade III or greater uterovaginal descent and corresponding symptoms. Complete procidentia was defined as uterovaginal eversion with descent of the uterine fundus past the hymenal ring. Controls had minimal or no apparent cystocele, rectocele or uterine prolapse on preoperative pelvic examination by a urogynecologist. The control patients underwent hysterectomy for uterine bleeding, premalignant cervical or uterine disease, or as part of a staging procedure during surgery for an adnexal mass, and denied symptoms referable to pelvic floor dysfunction.

An urogynecologist was present during removal of tissue specimens. During hysterectomy, after identification and transection of the uterosacral ligaments, 5 mm×1 cm of tissue was sharply excised between the transected margin of the ligament and the uterine cervix. Deep tissue of the ligament was separated from vaginal epithelium and peritoneum. In 11 subjects, 5 micrometer sections were stained with H&E, and microscopic examination confirmed uniform histology. A subset of 8 subjects with pelvic organ prolapse and 8 controls without prolapse were selected consecutively for genomic DNA measurements and are the subjects of the examples described herein.

DNA Isolation and Bisulfite Modification. Genomic DNA was isolated from the uterosacral ligament samples that were collected from each of the 8 subjects with pelvic organ prolapse (and from the uterosacral ligament samples that were collected from each of the 8 controls) using the Qiagen DNeasy® Tissue Kit according to the manufacturer's instructions. (Qiagen, Valencia, Calif.). One μg of DNA underwent sodium bisulfite modification using the EZ DNA Methylation-Gold™ kit (Zymo Research, Orange, Calif.) and was suspended in buffer solution to a final concentration of 10 mmol/L.

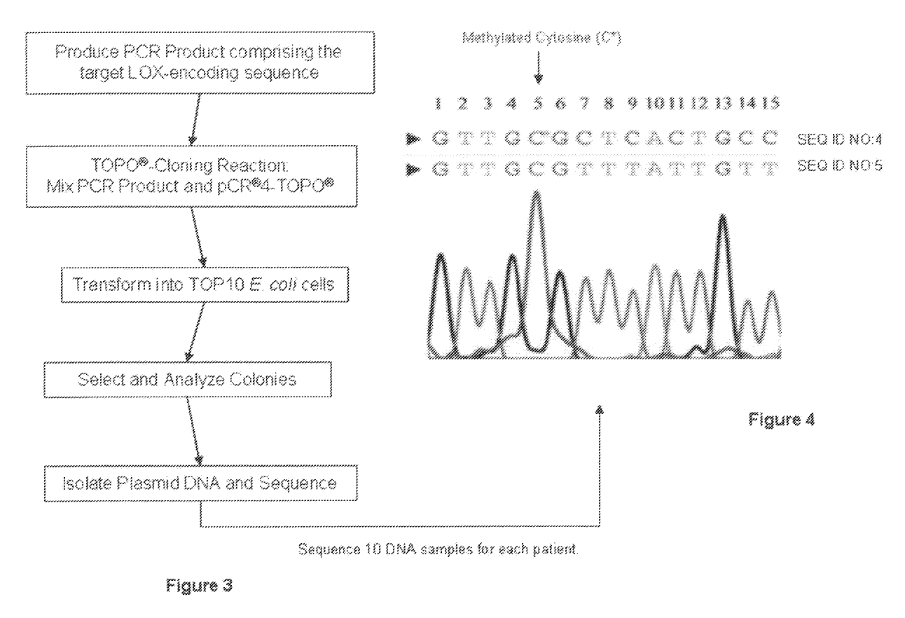

Bisulfite Sequencing. One μL of the sodium bisulfite-modified genomic DNA solution was used for reverse transcription-PCR (RT-PCR) to amplify the LOX promoter region of nucleotide −246 to +74 (SEQ ID NO: 1). The primers used were common to methylated and unmethylated DNA sequences, which are represented by SEQ ID NO: 4 and SEQ ID NO: 5. The resulting cDNAs were moved into the pCR2.1TOPO vector (Invitrogen, Carlsbad, Calif.) and plasmids were transformed into DH5α E. coli cells (Zymo Research, Orange, Calif.). E. coli cells resistant to ampicillin and kanamycin were cloned. Total DNA was then extracted from each set of cultured E. coli cells (i.e., 8 sets of E. coli cells harboring the LOX promoter region of control samples (patients without prolapse); and 8 sets of E. coli cells harboring the LOX promoter region of test samples (patients with prolapse)). The LOX promoter region of 10 DNA samples extracted from each of the control and test samples was sequenced. (Applied Biosystems, Foster City, Calif.). Methylated CpG islands were then identified by sequence analysis comparison—between test and control samples. The foregoing process is generally outlined in FIG. 3.

Results.

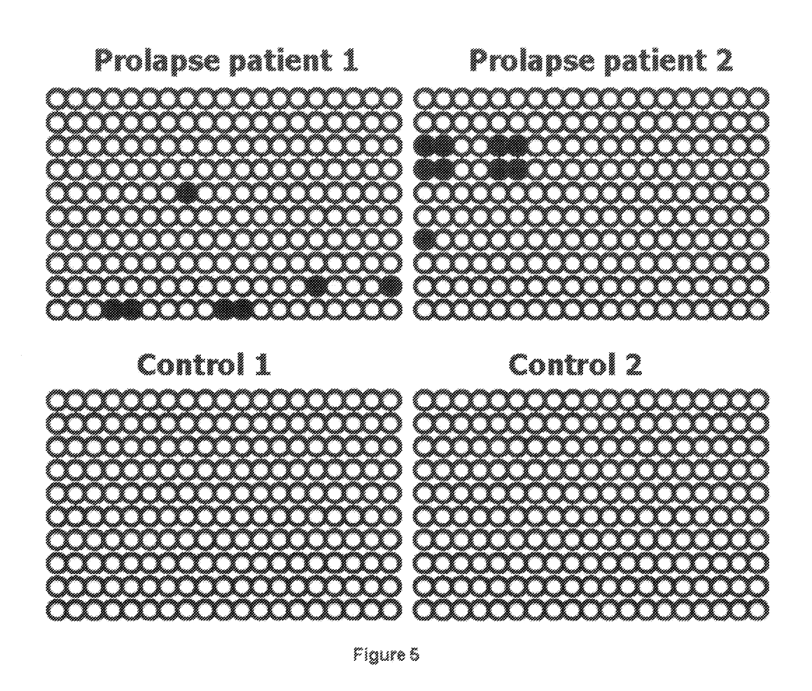

FIG. 4 provides a non-limiting example of a limited portion of nucleic acid sequence data related to a promoter region operably linked to LOX-encoding sequence. FIG. 5 is a graphical representation of the sequence comparison between the promoter region operably linked to a LOX-encoding sequence in a plurality of test samples and control samples—derived from two separate test and control patients. The darkened circles (wells) comprise DNA that includes a promoter region operably linked to a LOX-encoding sequence that was determined to contain methylated CpG islands. As shown in FIG. 5, the promoter region operably linked to a LOX-encoding sequence within the test samples (DNA derived from patients with prolapse) was found to have significantly more methylated CpG islands than that of the control patients (which, for the two control patients represented in FIG. 5, were found not to have any such methylated CpG islands). More specifically, among the test (prolapse-positive) samples analyzed, 66 methylated CpG islands were identified, whereas only 1 methylated CpG island was identified in the control (prolapse-negative) samples.

The invention provides that methylation in the promoter region operably linked to a LOX-encoding sequence suppresses LOX gene expression. As such, the invention provides that the presence of methylated CpG islands within such promoter region is indicative that a patient may have, or does have, a medical condition related to connective tissue failure or dysfunction, such as pelvic floor dysfunction, genital prolapse, and/or similar medical conditions.

Although illustrative embodiments of the present invention have been described herein, it should be understood that the invention is not limited to those described, and that various other changes or modifications may be made by one skilled in the art without departing from the scope or spirit of the invention.

What is claimed is:

1. A method of diagnosing a medical condition related to connective tissue failure or dysfunction, which comprises the steps of:
(a) extracting total genomic DNA from a specimen collected from a patient;
(b) amplifying a promoter region operably connected to a nucleic acid sequence encoding a protein selected from the group consisting of lysyl oxidase (LOX), lysyl oxidase like-1 (LOXL1), lysyl oxidase like-2 (LOXL2), lysyl oxidase like-3 (LOXL3), and lysyl oxidase like-4 (LOXL4); and
(c) determining whether the promoter region comprises methylated CpG islands.

2. The method of claim 1, wherein the medical condition is (a) genital prolapse, (b) pelvic floor dysfunction, (c) aortic aneurysm, or (d) a predisposition to developing any of the foregoing.

3. The method of claim 1, wherein the total genomic DNA is extracted from uterosacral ligament tissue, cells collected through vaginal scraping, a patient's whole blood, or a combination of the foregoing.

4. The method of claim 1, wherein determining whether the promoter region comprises methylated CpG islands is performed by sequencing the promoter region to obtain a set of test sequence data and comparing the test sequence data to a set of control sequence data, wherein the control sequence data represent a nucleic acid sequence of a LOX promoter that does not contain methylated CpG islands.

5. The method of claim 1, wherein the promoter region is operably connected to a nucleic acid sequence that encodes LOX.

6. The method of claim 1, wherein the promoter region is operably connected to a nucleic acid sequence that encodes LOXL1.

7. The method of claim 1, wherein the promoter region comprises a nucleic acid sequence that is substantially similar to SEQ ID NO: 1.

8. The method of claim 1, wherein the promoter region comprises a nucleic acid sequence represented by SEQ ID NO:1.

9. The method of claim 1, wherein the promoter region is amplified by PCR using two primer sequences represented by SEQ ID NO:2 and SEQ ID NO:3.

10. A method of preventing or ameliorating the effects of genital prolapse or pelvic floor dysfunction, which comprises the steps of:
(a) extracting total genomic DNA from uterosacral ligament tissue, cells collected through vaginal scraping, a patient's whole blood, or a combination of the foregoing;
(b) amplifying a promoter region operably connected to a nucleic acid sequence encoding a protein selected from the group consisting of LOX, LOXL1, LOXL2, LOXL3, and LOXL4;
(c) determining whether the promoter region comprises methylated CpG islands; and
(d) if the promoter region comprises methylated CpG islands, prescribing a preventative action to the patient.

11. The method of claim 10, wherein determining whether the promoter region comprises methylated CpG islands is performed by sequencing the promoter region to obtain a set of test sequence data and comparing the test sequence data to a set of control sequence data, wherein the control sequence data represent a nucleic acid sequence of a LOX promoter that does not contain methylated CpG islands.

12. The method of claim 10, wherein the promoter region is operably connected to a nucleic acid sequence that encodes LOX.

13. The method of claim 10, wherein the promoter region is operably connected to a nucleic acid sequence that encodes LOXL1.

14. The method of claim 10, wherein the promoter region comprises a nucleic acid sequence that is substantially similar to SEQ ID NO:1.

15. The method of claim 10, wherein the promoter region comprises a nucleic acid sequence represented by SEQ ID NO:1.

16. The method of claim 10, wherein the promoter region is amplified by PCR using two primer sequences represented by SEQ ID NO:2 and SEQ ID NO:3.

17. A method of diagnosing genital prolapse, pelvic floor dysfunction, or a predisposition to developing either of the foregoing, which comprises the steps of:
(a) extracting total genomic DNA from uterosacral ligament tissue, cells collected through vaginal scraping, a patient's whole blood, or a combination of the foregoing;
(b) amplifying a promoter region, which comprises a nucleic acid sequence represented by SEQ ID NO:1 and is operably connected to a nucleic acid sequence encoding a protein selected from the group consisting of LOX, LOXL1, LOXL2, LOXL3, and LOXL4, by PCR using two primer sequences represented by SEQ ID NO:2 and SEQ ID NO:3; and
(c) determining whether the promoter region comprises methylated CpG islands by sequencing the promoter region to obtain a set of test sequence data and comparing the test sequence data to a set of control sequence data, wherein the control sequence data represent a nucleic acid sequence of a LOX promoter that does not contain methylated CpG islands.

18. The method of claim 17, wherein the promoter region is operably connected to a nucleic acid sequence that encodes LOX.

19. The method of claim 17, wherein the promoter region is operably connected to a nucleic acid sequence that encodes LOXL1.


SEQUENCE LISTING (5 sequences)

SEQ ID NO: 1 - DNA, 1574 bp, Homo sapiens
cgccgctccccgttgccttccaggactgagaaaggggaaagggaagggtgccacgtccgagcagccgccttgactggggaagggtctgaatcccacccttggcattgcttggtggagactgagatacccgtgctccgctcgcctccttggttgaagatttctccttccctcacgtgatttgagccccgtttttattttctgtgagccacgtcctcctcgagcggggtcaatctggcaaaaggagtgatgcgcttcgcctggaccgtgctcctgctcgggcctttgcagctctgcgcgctagtgcactgcgcccctcccgccgccggccaacagcagcccccgcgcgagccgccggcggctccgggcgcctggcgccagcagatccaatgggagaacaacgggcaggtgttcagcttgctgagcctgggctcacagtaccagcctcagcgccgccgggacccgggcgccgccgtccctggtgcagccaacgcctccgcccagcagccccgcactccgatcctgctgatccgcgacaaccgcaccgccgcggcgcgaacgcggacggccggctcatctggagtcaccgctggccgccccaggcccaccgcccgtcactggttccaagctggctactcgacatctagagcccgcgaagctggcgcctcgcgcgcggagaaccagacagcgccgggagaagttcctgcgctcagtaacctgcggccgcccagccgcgtggacggcatggtgggcgacgacccttacaacccctacaagtactctgacgacaacccttattacaactactacgatacttatgaaaggcccagacctgggggcaggtaccggcccggatacggcactggctacttccagtacggtctcccagacctggtggccgacccctactacatccaggcgtccacgtacgtgcagaagatgtccatgtacaacctgagatgcgcggcggaggaaaactgtctggccagtacagcatacagggcagatgtcagagattatgatcacagggtgctgctcagatttccccaaagagtgaaaaaccaagggacatcagatttcttacccagccgaccaagatattcctgggaatggcacagttgtcatcaacattaccacagtatggatgagtttagccactatgacctgcttgatgccaacacccagaggagagtggctgaaggccacaaagcaagtttctgtcttgaagacacatcctgtgactatggctaccacaggcgatttgcatgtactgcacacacacagggattgagtcctggctgttatgatacctatggtgcagacatagactgccagtggattgatattacagatgtaaaacctggaaactatatcctaaaggtcagtgtaaaccccagctacctggttcctgaatctgactataccaacaatgttgtgcgctgtgacattcgctacacaggacatcatgcgtatgcctcaggctgcacaatttcaccgtattagaaggcaaagcaaaactcccaatggataaatcagtgcctggtgttctgaagtgggaaaaaatagactaacttcag

SEQ ID NO: 2 - DNA, 26 bp, Artificial
  PCR Primer
aagttagtgtgttttaggatgtgtgt

SEQ ID NO: 3 - DNA, 22 bp, Artificial
  PCR Primer
cttccctttcccctttctcaat

SEQ ID NO: 4 - DNA, 26 bp, Artificial
  PCR Primer
aagttagtgtgttttaggatgtgtgt

SEQ ID NO: 5 - DNA, 22 bp, Artificial
  PCR Primer
cttccctttcccctttctcaat